(19) United States
(12) Patent Application Publication
(10) Pub. No.: US 2006/0224146 A1
(43) Pub. Date: Oct. 5, 2006

(54) Method and system for non-invasive treatment of hyperopia, presbyopia and glaucoma

(76) Inventor: J. T. Lin, Oviedo, FL (US)

Correspondence Address: J. T. Lin, 4532 Old Carriage Trail, Oviedo, FL, 32765, US

(21) Appl. No.: 11/206,853

(22) Filed: Aug. 19, 2005

Related U.S. Application Data
(63) Continuation-in-part of application No. 11/092,662, filed on Mar. 30, 2005.

(51) Int. Cl. A61B 18/18

(52) U.S. Cl. 606/4

(57) ABSTRACT

Laser and non-laser means for selective thermal shrinkage of ocular tissue (including cornea, sclera, choroids and ciliary-body) for the treatment of hyperopia, presbyopia and glaucoma are disclosed. The preferred system includes lasers in visible (0.48 to 0.78 micron) and IR (1.4 to 2.2 micron), and non-laser device of radio frequency wave including electrode device, bipolar device and plasma-assisted device. Two predetermined treated area having a circle diameter of about (6 to 8) mm and about (10 to 14) mm are defined. A revised Beer's law is introduced, Bexp(−dA), to relate the focusing factor (B), penetration depth (d) and the absorption coefficient (A) at a given laser spectra. An optimal focal length about 0.8 to 1.4 times of (InB*)/A is formulated for lens design. The effective thermal penetration depth, d*=(0.3−1.0) mm, may be achieved by choosing an optimal focal length laser, or by the length of the conductor tip (about 0.45 to 1.2 mm) of the radio frequency device.

### RELATED APPLICATION

This application is a Continuation-in-part of U.S. application Ser. No. 11/092,662 filed on Mar. 30, 2005, the teachings of which are incorporated herein by this reference in their entirety.

### BACKGROUND OF THE INVENTION

1. Field of the Invention

The present invention relates to method and apparatus for non-invasive treatment of eye disorders of hyperopia, presbyopia and glaucoma by using a thermal energy beam (laser or radio frequency wave) to reshape the corneal surface, or increase the accommodation or lower the intraocular pressure of treated eye.

2. Prior Art

Corneal reshaping including procedures of photorefractive keratectomy (PRK) and laser assisted in situ keratomileusis (LASIK) have been performed by lasers in the ultraviolet (UV) wavelength of (193-213) nm. The commercial UV refractive lasers include ArF excimer laser (at 193 nm) in U.S. Pat. No. 4,773,414 of L'Esperance, et al. and non-excimer, solid-state lasers such as those proposed by the present inventor in 1992 (U.S. Pat. No. 5,144,630) and in 1996 (U.S. Pat. No. 5,520,679). The above-described prior arts using lasers to reshape the corneal surface curvature, however, are limited to the corrections of myopia, hyperopia and astigmatism and exclude the treatments of presbyopia or glaucoma.

Refractive surgery using a scanning device and lasers in the mid-infrared (mid-IR) wavelength was first proposed by the present inventor in U.S. Pat. Nos. 5,144,630 and 5,520,679 and later proposed by Telfair et al. in U.S. Pat. No. 5,782,822, where the generation of mid-IR wavelength of (2.5-3.2) microns were disclosed by various methods including: the Er:YAG laser (at 2.94 microns), the Ramar-shifted solid-state lasers (at 2.7-3.2 microns) and the optical parametric oscillation (OPO) lasers (at 2.7-3.2 microns).

Corneal reshaping may also be performed by thermal shrinkage using a Ho:YAG or diode laser (at about 2 microns in wavelength), disclosed by Sand in U.S. Pat. No. 5,484,432, a procedure known as Ho:YAG laser thermal keratoplasty (HLTK); or by a diode laser thermal keratoplasty (DTK); or by a procedure called conductive keratoplasty (CK) using a radio frequency (RF) wave, for example, device disclosed by Doss and Hutson in U.S. Pat. Nos. 4,326,529 and 4,381,007. These methods, however, were limited to low-diopter hyperopic corrections. Strictly speaking, these prior arts cannot be used to correct the true “presbyopia” and only performed the mono-vision for hyperopic patients. A thermal beam (or energy) is required in these prior arts and the treated area is inside the limbus; within the optical zone diameters of about 6 to 8 mm. Because the corneal surface is reshaped, the treated eye of presbyopia will lose its far vision while it is a “overcorrected” for slightly myopic to see near.

Prior art of Ruitz (U.S. Pat. No. 5,533,997) proposed the use of ArF excimer laser for presbyopia by multifocal effect which again involves with corneal surface reshaping in the central optical zone area.

The above prior arts, therefore, did not actually resolve the intrinsic problems of presbyopic patient caused by age where the lens loses its accommodation as a result of loss of elasticity in ciliary-body or scleral layer due to age.

All of the above-described prior arts used methods to change the cornea surface curvature either by tissue ablation (such as in UV laser LASIK) or by thermal shrinkage (such as in HLTK, DTK and CK) are limited to the cornea, about 6 to 8 mm diameter area. In contract, an area outside the limbus about 10 to 14 mm is treated in presbyopia correction disclosed in this invention. The non-contact mode used in the prior art of HLTK suffers major regression due to its limited penetration depth of the laser energy (less than about 0.2 mm). Contact mode used in conventional DTK and penetrating needle used in CK may improve the stability, however, they still suffer poor predictability postoperative major regression and initial efficacy of these prior arts limited their application only for low hyperopia correction over the non-dominant eye. The prior art of Sand (HLTK) disclosed a preferred short pulse (about 10 milliseconds) laser at about 1.8 to 2.2 micron with an exposure time about 0.1 second and operated at non-contact, non-focused mode. In contrast, one of the preferred embodiment of the present patent is to use a CW diode laser (at about 1.4 to 1.9 microns) operated at a contact, focused mode with an exposure time about 2 to 5 seconds, where deeper penetration of laser energy is achieved by optimal focusing for more stable and predictable results than HLTK.

In the prior arts of HLTK, conventional DTK or CK for the treatment of hyperopia, the treated area is within the cornea area (defined as one-zone method) in comparing to the two-zone method which also includes the second zone in the sclera area (outside the limbus) as proposed in the present invention. Higher hyperopia (up to about 5 diopter) correction is possible using the two-zone method proposed in this invention, where thermal energy is applied on both the cornea and sclera area. Furthermore, prior arts using one-zone method suffered major postoperative regression due to shallow penetration and poor predictability of refractive outcome due to the non-controlled spot size and absorption coefficient (A). For example, A has a wide range of 30 to 70 1/cm, for a laser spectral of 1.8 to 2.2 microns disclosed by the prior art of Sand. Without specifying these spectra, within a narrow range of less than 0.01 micron, the uncertainty of A will result in unknown penetration depth which is critical in the outcome. Greater details will be shown later.

The direct method for presbyopia correction is to increase the accommodation of the presbyopic patients by changing the intrinsic properties of the sclera or ciliary tissue to increase the lens accommodation without changing the corneal curvature. Because there is no reshaping of the cornea, the treated eye shall keep is original far vision while its near vision is improved under a presbyopia treatment. This is the fundamental difference between corneal reshaping and the change of sclera-ciliary tissue property.

To treat presbyopic patients using the concept of expanding the sclera by sclera expansion band (SEB) was proposed by Schachar in U.S. Pat. Nos. 5,489,299, 5,722,952, 5,465,737 and 5,354,331. The mechanical SEB approach has the drawbacks of complexity, major invasive, time consuming, costly, potential side effects and with major postoperative regression. To treat presbyopia, the Schachar U.S. Pat. Nos. 5,529,076 and 5,722,952 proposed the use of heat or radiation on the corneal epithelium to arrest the growth of the crystalline lens or deliver heat to the sclera or zonules. However, there were no parameters specified for the source of heat or radiation. No laser device was made and no clinical studies have been conducted to show the effectiveness of the concepts proposed by Schachar over 10 years ago.

Schachar's prior arts simply included all the available “names” of lasers picked from textbooks, without specifying their difference in response to tissue absorption. Names of lasers should not be patented. As shown in the present invention, localized, selected heating of soft tissues by a laser requires specific laser parameters and the tissue absorption properties in response to a laser at a given spectrum are the critical elements. Without specifying these elements, Schachar's concept will fail in any practical system or procedure. Furthermore, the lack of information on clinical issues, such as locations, patterns and depth of the treated tissue also prevents any clinically useful system to be made based on Schachar's prior arts.

The prior art of Bille (U.S. Pat. No. 4,907,586) proposed the use of picoseconds short pulse laser focused directly to the lens of an eye for presbyopia treatment. This method, however, has never been clinically tested due to the risk of cataract and technical difficulties in laser spot size position control. This prior art was also limited to laser specifications of pulse duration less than 10 picoseconds, energy per pulse less than 30 micro joule. This prior art uses laser to rupture tissue and will not produce the thermal shrinkage required in the present invention.

The prior arts of the present inventor, U.S. Pat. No. 6,258,082, 6,263,879, 6,824,540 and PCT/US01/24618 (together defined as “Lin-62-68”) proposed the use of a laser to remove a portion of the sclera tissue based on the concept of “lens relaxation”, where the scleral ablation causes the ciliary body to contract for lens relaxation to see near. From our clinical results using the method proposed in our prior arts, we found that there are two major drawbacks: first, regression is improved (less than that of incision method and SEB), but still significantly reduce the efficacy for postoperation after 9 to 12 months; secondly, the initial accommodation amplitude (AA) ranging from 0.5 to 2.5 diopter (with a mean about 1.9 diopter) is too low when postoperative regression of (20% -40%) is included. In addition, our clinical data also showed the total failure in some cases, where the accommodation amplitude (AA) after surgery is less than 0.5 diopter with Jaeger (J) reading higher than 5. The acceptable J-reading is J=(1.0 to 3.0) for near vision at about 40 cm. A successful treatment for typical patients shall reduce the preoperative J-reading (about 5 to 7) such that a Snellen near value of 20/32 (or J3) or better is achieved. For severe presbyopia with preoperative J=(10 to 15), a successful treatment shall expect J=(3 to 5), postoperatively. If minor regression of (5% to 15%) is allowed, a successful treatment will require an initial AA of about (1.8 to 3.5) diopters.

The prior arts of Lin-62-68 failed to meet the above criteria for those cases with regressions or those cases with lower initial AA (say, less than 1.2 diopter) after laser sclera ablation. They are also highly invasive surgical methods in comparing to the non-invasive, non-ablative thermal method of this invention. Furthermore, these prior arts require the presbyopia patient to have a normal far vision with hyperopia than 1.0 diopter. Patient with hyperopia must be corrected by LASIK, HLTK or CK before the treatment. In comparison, the teaching disclosed in the present invention will treat both hyperopia and presbyopia when the two-zone method is used.

Prior art of Lin's and Martin's, U.S. Pat. No. 6,491,688, proposed a non-invasive method using a gonio lens guided infrared laser to heat the zonules fiber of the eye for the treatment of presbyopia. This prior art, however, suffers both clinical and technological difficulties. It is very difficult to control the gonio lens angle for a laser to target at zonules while keeping the lens and iris intact. The clinical outcome and potential complications of laser thermal shrinkage of zonules have not been tested. In addition, the selected heating of zonules is limited by the transparency of cornea and humous cavity at the selected laser spectra.

It was previously known, for example, in: Bargeon et al., “Calculated and measured endothelial temperature histories of excised rabbit cornea explored to IR radiation”, (Exp. Eye Res. Vol. 32, 241-250, 1981); and Stringer et al., “Shrinkage temperature of eye collagen”, (Nature, vol. 204, p. 1307, 1964); that collagen fiber may contract to about ⅓ of their linear dimension, when it is heated to about 58 to 75 degree Celsius.

Radio frequency (RF) wave had been also commercially used for the treatment of snoring by thermal shrinkage of the throat soft tissues since 1996. More recently, RF was used in the procedure of CK as described earlier. The thermal energy procedures for corneal shrinkage, HLTK, conventional DTK and CK, all are limited to the treatment of low hyperopia, and limited to the treatment of non dominant single eye of presbyopic patient. These prior arts can not treat both eyes since the dominant eye must remain for far vision. In contrast, the present invention discloses methods to treat both eyes of presbyopic patient to see near, whereas their far vision remains. In addition, there is a strong need to treat patients having both hyperopia and presbyopia, which is not available so far.

There are commercially available lasers, such as a green Nd:YAG, for the treatment of retina diseases. However, there is no system available for the treatment of presbyopia or glaucoma using either thermal lasers or RF wave applied to the sclera, choroids or ciliary body as proposed in the present invention.

One objective of the present invention, therefore, is to provide an apparatus and method to obviate the drawbacks in the prior arts. In particular, a procedure which is non-invasive, no bleeding, fast tissue healing, safer and no tissue ablation (a non-surgical procedure).

It is yet another objective of the present invention to provide method and system having improved efficacy for presbyopia treatment by “thermal shrinkage” of the conjunctiva, sclera, choroids or ciliary body, rather than “ablation” of sclera or ciliary proposed by Lin's prior arts.

It is yet another objective of the present invention to provide the optimal parameters of the thermal energy beam (laser or RF wave) for sufficient thermal shrinkage of the treated ocular tissues. These parameters include beam power, spot size control, penetration depth, location and pattern of the treated area, and configuration of system optics and energy delivery.

It is yet another objective of the present invention to provide a revised Beer's law for lens design and for optimal thermal penetration of the treated tissue. This formula relates the tissue absorption coefficient, laser wavelength, spot size, laser power density and penetration depth which are the critical elements for stable and predictable outcome.

It is yet another objective of the present invention to provide a method and system which can treat hyperopia, presbyopia, or glaucoma, or combined treatment of above for both eyes.

A further objective is to provide a treatment for hyperopic, aged patient who requires both hyperopia and presbyopia corrections.

A further objective is to provide a treatment for hyperopia, presbyopia, where the thermal shrinkage induces accommodation may be further enhanced by accumulated transient electrical or thermal stimulation in the ciliary body or zonules.

It is yet another objective of the present invention is that outflow of the vitreous is improved after the procedure to reduce the abnormally high intraocular pressure (IOP) of primary open angle glaucoma patients.

Further objectives of the invention will become apparent from the description of the invention to be detailed as follows.

### SUMMARY OF THE INVENTION

A two-component theory consisting of crystalline lens relaxation (or surface curvature change) and its anterior shift is needed for maximal accommodation. Ciliary body (CB) contraction may be enhanced by either increase the elasticity or spacing of the scIera-ciliary-zonule complex by a thermal energy applied to the complex. The preferred tissue heating means include laser and non-laser energy. The preferred two-zone method includes localized heating of (1) area outside the limbus and on the soft tissue of sclera, choroids or ciliary-body of the eye for presbyopia correction; and (2) corneal surface area of about 6 to 8 mm in diameter for hyperopia correction.

It is yet another preferred embodiment is that CB or choroids layer is selectively heated with minimal heating of the conjunctiva layer or sclera layer, where the localized temperature is raised to about 55 to 85 degrees Celsius, most preferable about 58 to 75 degree Celsius and causes efficient thermal shrinkage after the treatment, such that CB contraction is enhanced for greater accommodation.

It is yet another preferred embodiment includes the heating pattern on the treated area having a minimal of 4 spots, preferable 8 to 32 spots, symmetrically around a circumference of a circle having a diameter about 6 to 8 mm (for hyperopia correction) or about 10 to 14 mm (for presbyopia correction).

It is yet another preferred embodiment is that the spot size at the treated surface is about 0.8 to 2.0 mm when a laser is used; and about 0.1 to 0.3 mm when a RF wave is used.

It is yet another preferred embodiment is that the spot size and penetration depth (d) are controlled by the design of mini-lens calculated by a revised Beer's law given by Bexp(−dA), where B is a focusing factor, A is the tissue absorption coefficient.

It is yet another preferred embodiment is to use medication such as pilocarpine or medicines with similar nature that triggers ciliary body contraction to stabilize and/or enhance the post-operative results. A further preferred embodiment is to provide a treatment for hyperopia, presbyopia, where the thermal shrinkage induces accommodation may be further enhanced by accumulated transient electrical or thermal stimulation in the ciliary body or zonules.

It is yet another preferred embodiment includes the use of a fiber-delivered laser beam having a focusing lens at the tissue contact tip for maximal penetration depth of the thermal energy about 0.4 to 1.5 mm depending on the absorption coefficient of the treated tissue (A). The preferred focal length (f) of the lens includes f=(0.8 to 2.0) mm for A=(20-55) cm⁻¹ and f=(0.3-0.8) mm, for A=(56-70) cm⁻¹, where the laser spot size at the focal point includes about 0.08 to 0.5 mm, most preferable about 0.1 to 0.3 mm.

It is yet another preferred embodiment that the thermal energy beam includes lasers in infrared about 1.4 to 2.2 microns, most preferable about (1400 to 1500) nm, (1875 to 1890)nm and (2000 to 2150) nm for the treatment of cornea or sclera tissue; and visible lasers of about 0.48 to 0.78 microns for the treatment of choroids or ciliary body. The non-laser sources of radio frequency (RF) wave, such as electrode device, bipolar device or plasma-assisted electrode, having a RF frequency about 200 KHz to 500 KHz; and power of about 100 to 600 mW per spot of laser or RF energy at the treated area.

Further preferred embodiments of the present invention will become apparent from the description of the invention that follows.

### BRIEF DESCRIPTION OF THE DRAWINGS

FIG. 1 The ocular structure of a human eye (a side view).

FIG. 2 Laser power density profiles for a focused and non-focused laser beam at various absorption coefficient and depth.

FIG. 3 Temperature profiles at various focal point.

FIG. 4 Penetration depth and thermal patterns.

FIG. 5 The preferred system and lens design for various laser beam spot size and penetration profile.

FIG. 6 Structure of hand piece of radio frequency device.

FIG. 7 The preferred embodiments using electrode needle of radio frequency device.

### DETAILED DESCRIPTION OF THE INVENTION AND THE PREFERRED EMBODIMENTS

An ophthalmic system in accordance with the present invention comprises a tissue heating means or thermal energy beam, including electromagnetic wave such as a coherent wave (or laser), and non-laser wave such as radio frequency (RF) wave used in electrode device, bipolar device and plasma assisted electrode device.

Radio Frequency (RF) Device

When a RF device is used, the preferred embodiment requires a minimum thermal energy (or current or power) to the treated tissue with efficient thermal shrinkage which is further defined by a preferred frequency about 100 KHz to 800 KHz, most preferable about 200 to 500 KHz. The preferred RF generator current is modulated for coagulation with an output power about 0.5 to 5 W and about 0.1 to 0.8 W for each of the treated spot depending on the areas treated soft tissues of the eye. The preferred treating period of each spot is about 0.2 to 2.0 seconds. The dimension of the heated tissue (depth, width and length) is controlled by the power, peak voltage and size of the RF energy beam. The penetration depth of the RF energy may also be controlled by the size, length and structure of the electrode tip which is inserted into the treated area (to be detailed later). The preferred RF electric current of about 100 KHz to 800 KHz and the treatment period (for each spot) of the present invention are much smaller than that of prior arts of Doss (U.S. Pat. No. 6,326,529 and 4,381,007), who proposed a typical value of 2,000 KHz and a duration of 1 to 10 seconds. Furthermore, prior arts of Doss require the use of coolant to control deep thermal penetration, whereas no coolant is needed in this invention and thermal depth is controlled by the structure and penetration depth of the electrode.

Thermal Lasers

When a laser is used, we also require efficient localized tissue heating with minimal thermal damage to the non-treated tissue. Therefore, the preferred laser spectrum is the region where ocular tissues (containing blood, melanin, protein or water) have certain absorption, but not too strong, in order to penetrate deep into the treated area without significantly heating the conjunctival or surface layer. We also note that laser absorption is largely by protein and water in cornea conjunctiva and sclera, and water, melanin and red blood cells (hemoglobin) in choroids and ciliary body. Based on these criteria, the preferred laser spectrum includes visible lasers about 0.48 to 0.78 micron, for thermal shrinkage choroids or ciliary body; and infrared (IR) laser at about 1.4 to 2.2 micron for thermal shrinkage of cornea, conjunctiva or sclera. Other ranges of spectrum with very strong tissue absorption such as IR laser of (2.8 to 3.2) microns, or UV laser of (193 to 300) nm should be excluded. These “ablation-type” lasers, excluded in the present invention, are required in the prior arts of Lin. For lasers in the above selected visible, UV or IR range, the preferred pulsed duration is longer than 500 microseconds, or a continuous wave (CW) mode at low peak power. These long pulse requirement is also excluded in prior arts of Lin in ablation procedures.

Therefore, the preferred lasers shall include solid-state at about 1.4 to 2.2 microns, such as Ho:YAG (at about 2.1 micron), Nd:glass (at 1.54 micron), diode-laser-pumped fiber laser (at about 1.4 to 1.5 micron), Nd:YAG (at 1.4 micron) and semiconductor diode lasers (at 0.63 to 0.78 or 1.4 to 1.9 microns); visible solid-state laser of harmonic of Nd:YAG or Nd:YLF (at 532 or 526 nm); argon-ion laser at 488 to 514 nm, HeNe laser at 633 nm; krypton-ion at 647 nm; He—Cd laser at 442 nm; dye lasers at (0.5 to 0.7) microns. The most preferable laser spectra are at about 0.488, 0.53, 0.58, 0.63, 0.67, 0.75, 1.4, 1.45, 1.54, 1.89 and 2.1 microns. The preferred laser pulse width is longer than 500 microsecond, or a continuous wave (CW) laser. Green laser from frequency-doubled Nd:YAG, Nd:YLF or Nd:YVO4 may also be used, when choroids or ciliary body is the treated area, where energy per pulse shall be less than 5 mJ. The preferred laser beam spot size (for non-contact mode) or the size of the fiber tip (for contact mode) is about (0.2 to 1.0) mm at the treated surface. And the preferred laser power for each of the treated spot is about (0.1 to 1.0) W, depending on spot size, and spectrum of the laser beam.

The prior art of Sand (HLTK) proposed a preferred short pulse (about 10 millisecond) laser with an exposure time about 0.1 second and operated at non-contact and non-focused mode. In contrast, one of the preferred embodiment of the present invention is to use a CW diode laser (at about 1.4 to 1.9 microns) operated at a contact and focused mode with an exposure time about 2 to 5 seconds, where deeper penetration of laser energy is achieved for more predictable and stable results than HLTK.

In the prior arts of HLTK, DTK or CK for the treatment of hyperopia, the treated area is in the cornea area defined as one-zone method in comparing to the two-zone method which also includes the second zone in the sclera area (outside the limbus) as proposed in the present invention. Therefore, higher hyperopia (up to about 5 diopter) correction is possible using the two-zone method, where thermal energy is applied on both cornea and sclera area.

Accommodation for Presbyopia

We shall first describe our theory behind the invention for the increase of accommodation amplitude (M) to treat presbyopia. It has been known that presbyopia was caused by age. However, the complete description for the mechanisms of accommodation is not conclusive, which includes capsular theory (von Helmholtz theory), lens-crowding theory (Schachar theory), Catenary theory (by Coleman, Ophthalmology, 2001, vol. 108, page 1544-51) and the two-component elastic theory (by the present inventor, in J. Refractive Surgery, 2005, vol. 21, p. 200-201). All those theories, however, have a common principle that ciliary theory (CB) contraction causes lens accommodation for near vision, although lens power increase (or curvature change) may attribute to various mechanisms: via measure gradient between vitreous and aqueous compartments (Coleman), via relaxation of the lens capsule (Helmoholtz), or via combination of lens relaxation and anterior shift (Lin). Therefore, the fundamental issue for presbyopia treatment becomes how to improve or enhance the CB contraction which causes the increase of system power or lens power. Both the prior arts of SEB (Schachar) and laser scleral ablation (Lin) are dealing with the superficial layer of the sclera tissue, by sclera expansion or by increasing the elasticity of the laser ablated scleral regions. The change of scleral structure is then translated to the movement (or contraction) of the ciliary body (CB) and the zonular fiber connected to the lens.

Based on prior arts of Schachar's and Lin's, the influence from sclera superficial change to CB, then to zonules and lens is rather inefficient due to the ocular structure of the CB-zonule-lens complex, and the “remote” distance from the sclera layer to the zonule and lens. In addition, these prior arts are rather invasive surgical procedures taking about (15-30) minutes per eye. The present invention proposes to “directly” change the property of the CB tissue which is closer to the zonules-lens and therefore will be much more efficient. It was reported in a “stretching experiment” (Glasser and Campbell, Vision Research, vol. 38, p. 209-229) that each one mm contraction of CB may induce (0.8 to 2.6) diopter of accommodation in young lens (age 10-53), but almost no power change for old lens (age 54 to 87). This clinical data also supports our theory that lens anterior chamber shift (ACS) or axial movement shall play an important role, particularly in old eyes. The clinical data for presbyopia age (40-65) years also supports our theory that ACS shall play an important role, particularly in old eyes.

To calculate the total accommodation amplitude (AA), I propose the “Lin dynamic model” by introducing two components, the anterior shift (AS) or axial movement and lens relaxation (LR) M=AS+LR=M1(dS)+M2(dR), where lens power change is converted to AA by a factor, CF=(0.7-0.8), rather than 100% conversion. My calculations (Lin, Journal of Refractive Surgery, 2005, vol. 21, p. 200-201) showed that one mm dS of the lens will cause about M1=(1.0 to 1.7) diopter of myopic shift (for patients to see near) and the reversed process, posterior shift (PS) will allow the patient to see far. We note that these AS and PS are “dynamical” effects allowing the lens to move forward and backward for a presbyopic patient to accommodate both near and distance vision. The second component LR causes the presbyopic lens to see near by lens relaxation with decreased radii of the lens, mainly by the anterior capsule of the lens. For a typical post-surgery patients with an average accommodation amplitude (AA) of +2.0 D, I propose that the AA may attribute to AS or LR or the combination of them, depending on age of lens (or its rigidity).

The thermal heating method disclosed in the present invention has a fundamental difference with the prior arts of Lin (US patents Lin-62-68 defined earlier) in the mechanisms, location, depth and structure of the treated area. A thermal laser is needed in this invention, in contrast to the “cold” ablative laser needed in Lin's prior arts. The superficial scleral expansion (SEB of Schachar) or ablation (Lin-62-68) causes the increase of sciera ring radius, but it is very inefficient in affecting the CB contraction. These prior arts also suffer major regression due to sciera healing, as clinically reported.

Clinically, it is important to note that the total accommodation amplitude (AA) is governed by the amount of ciliary body contraction, therefore the AA shall be governed by the tissue property change after the treatment including the elasticity of the sclera or ciliary body (CB) tissue, the available space for CB contraction, or the distance between CB to the lens connected by the zonules fiber. In the proposed thermal shrinkage of the treated soft tissue (conjunctiva, sciera, choroids or ciliary-body) of this invention, there is a minimal amount of thermal energy needed in order to cause sufficient soft tissue shrinkage which is further governed by the localized temperature (T) of the treated tissue. Depending on the types of ocular soft tissue, the preferred T is about 50 to 85 degree Celsius (C), most preferable of 58 to 75 degree C. (based on data of Stringer et al, Nature, vol. 204, page 1307, 1964) and T shall not be too high to cause permanent tissue damage or evaporation. Given a thermal energy (E), T is proportional to the average power (in Watt) applied to the tissue W=Et, where t is the thermal energy beam (laser or RF wave) treating (exposure) time. When a laser is used, the preferred laser includes only those with energy can be localized absorbed by the treated tissue via the melanin, protein, blood or water content of the treated ocular tissue.

Two-Zone Method Using Laser

In addition, a minimal penetration depth of the thermal energy beam is required for efficacy and stable outcomes. For hyperopia correction, all the prior arts of HLTK, DTK or CK procedures are dealing with the area about 6 to 8 mm in diameter (or inside the limbus). For presbyopia correction of this invention, the treated area must be outside the limbus about 10 to 14 mm in diameter, in order to avoid thermal shrinkage of the cornea, while shrinkage occurs in the conjunctiva, sclera, choroids or ciliary-body. A deeper thermal depth (deep into the choroids or ciliary body tissue) about 0.5 to 1.2 mm is preferred in this invention for the treatment of presbyopia, in comparing to about 0.45 mm of prior arts for hyperopia correction. Deeper thermal depth is required for efficacy and stability of presbyopia treatment disclosed in this invention. Greater detail for lens design to achieve deep penetration will be discussed later.

The prior art of Sand (HLTK) disclosed the preferred short pulse (about 10 millisecond) laser with an exposure time about 0.1 second and operated at non-contact and non-focused mode. In contrast, one of the preferred embodiment of the present patent is to use a CW diode laser (at about 1.4 to 1.9 microns) operated at a contact or non-contact but focused mode with an exposure time about 2 to 5 seconds, where deeper penetration of laser energy via novel lens design is achieved for more stable results than HLTK.

In the prior arts of HLTK, DTK or CK for the treatment of hyperopia, the treated area is in the cornea area defined as one-zone method in comparing to the two-zone method also including the second zone in the sclera area (outside the limbus) as proposed in the present invention. Therefore, higher hyperopia (up to about 5 diopters) correction is possible using the two-zone method, where thermal energy is applied on both cornea and sclera area.

Another preferred mechanism of this invention is that electrical or thermal stimulation (ETS) of CB contraction (at a temperature much lower than the permanent shrinkage of about 55 degree C.) may also cause the loosening or increasing space of the SCZ complex, if the ETS is repeated and accumulated. For permanent shrinkage, a higher-power about 0.4 to 0.8 W (per treated spot) would be needed, in comparing to a preferred power about 0.05 to 0.2 W for the case of ETS. The ETS may be resulted from the thermal energy from a laser or a RF wave device.

Without the above clinical and theoretical analysis and specific lens design (to be detailed later), it would be very difficult to predict the clinical outcome. The method in this invention and parameters for the proposed device and clinical techniques are based upon the above theoretical findings. Further analysis on the mechanisms and efficiency of ciliary body contraction will be discussed and shown by figures as follows.

Thermal Shrinkage of Ocular Tissue

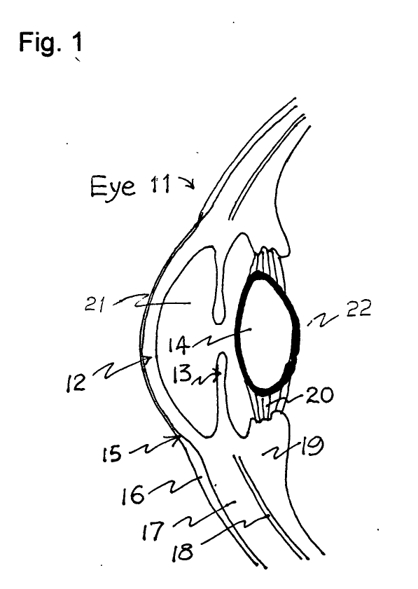

FIG. 1 shows the diagram of a human eye (a side view). The ocular structure of an eye 11 consists of the cornea 12, the iris 13, the lens 14, the limbus 15, the conjunctiva 16, the sclera 17, the choroid layer 18, and the ciliary body (CB) 19, which is connected to the lens 14 by the zonule 20. The lens shape and its location (or the anterior chamber depth) is governed by the tensile force from sclera-ciliary-zonule and the pressure (or pressure gradient) in the anterior chamber 21 and in the vitreous 22. The typical thickness of these ocular components is about: 0.5 to 0.7 mm for total thickness of conjunctiva, sciera and choroids layer; 0.6 to 1.4 mm for the thickness of CB having length about 4.5 to 5.5 mm; the limbus is located at about 5.0 to 5.5 mm from the center of the cornea. From this diagram, the following mechanisms are proposed for efficient CB contracting for accommodation. Accommodation amplitude (M) is given by a 2-component theory M=AS (anterior shift)+LR (lens relaxation). Both AS and LR are proportional to the amount of CB contraction (CBC) which is limited by the elasticity of the sclera-CB-zonule complex (SCZ) and the spacing (SP) among each of the SCZ components. Therefore “loosening” of sclera, CB or zonule will enhance CBC which is significantly reduced in aged eyes. To loosen this SCZ complex or increase their spacing, the present invention proposes to use thermal energy beam to shrink the complex, rather than tissue ablation in Lin's prior arts.

Based on the above described mechanisms, we are able to further analyze the efficiency of CB contraction (CBC). The amount of CBC is proportion to a momentum defined by P=MV=M(D/t), where M is the mass of the SCZ complex which moves at a speed of V (to move a distance D) during the accommodation or CB contraction period t. It was reported by Coleman et al. (Ophthalmology 2001, vol. 108, p. 1544-51) that the response time (t) was about 0.5 second in an electrical stimulation of CB of a primate eye for a rise of the vitreous pressure pulse. This stimulated CBC is transient and we look for a permanent capability of enhanced CBC. Using the concept disclosed in the present invention that CB contraction speed (V) or its distance (D) is inverse proportional to the mass of the SCZ complex (M) for a given momentum (P) or contraction force. Furthermore, increase of SCZ complex available spacing will also increase the speed V and its distance (d). For a given CB contraction force, the momentum also proportional to the elasticity of the SCZ complex. A thermal shrinkage of ocular tissue of sclera, choroids or CB will cause one or more than one of the above described effects which enhance the CBC. Note that about ⅓ deduction of the linear dimension of the tissue under thermal shrinkage is expected as previously known. This shrinkage will allow more anterior chamber shift of the lens or CBC, or both and therefore results in an increase of accommodation.

Another preferred mechanism of this invention is that electrical or thermal stimulation (ETS) of CBC (at a temperature much lower than the permanent shrinkage of about 65 degree C.) may cause the loosening or increasing space of the SCZ complex, if the ETS is repeated and accumulated. One preferred embodiment of this invention is to use the ETS as an enhancement of thermal shrinkage procedure. The preferred means of enhancement also includes the use of medicine such as pilocarpine (about 0.05% to 0.2%) or others in similar nature to trigger and enhance CBC. The preferred means of medicine enhancement can be applied before, during or after the procedure.

Laser Profile of Focused Beam

The laser power density (or irradiance) profile, normalized by its surface power density, of a focused laser inside an absorbing medium (ocular tissue) may be calculated by a revised Beers law (J. T. Lin, unpublished)
P=Bexp (−dA), (Eq. 1)
B=[f/(f−d)]², (Eq. 2)
where A is the absorption coefficient of the cornea tissue at a given laser spectrum; B is a focusing factor inverse proportional to the focused beam spot size(derived from simple geometry); f is the focal length of the optics (lens) and d is the penetration depth or position away from the corneal surface, d=0. It should be note that when d approaches f, B equals its maximal B* calculated by the square of the ratio between the initial beam spot size (at d=0) and the beam waist (a diffraction-limited finite size at d=f) to avoid the singularity of formula (2). From above formula, one may readily see that the laser irradiance has a fast exponential decrease due to its absorption in ocular tissue, which is competing with the increasing factor B in a focused beam. The net result of power absorption and beam spot decrease (or increase of power density) produces a peak value (P*) of the laser irradiance profile located at the focal point or position of minimal beam spot. For example, given a laser spot size (diameter) of 1.0 mm on the corneal surface (or d=0) which is focused to a minimal spot of 0.25 mm (at d=f), one may easily calculate, from Eq. 2, B=(1, 4, 16, 4, 1) at d/f=(0, 0.5, 1.0, 1.5, 2.0).

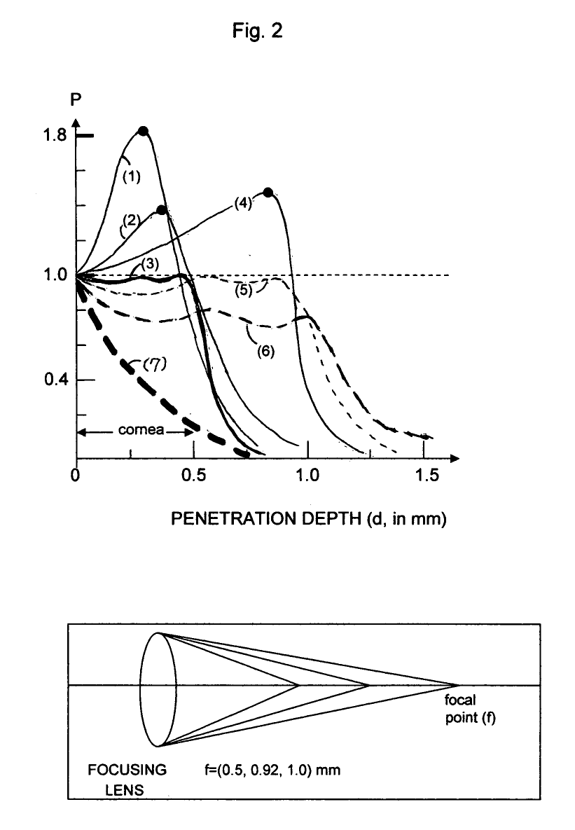

Using the published transmittance data (Atchinson and Smith, “Optics of human eye, p. 108) to calculate the absorption coefficient (A) to be about 20 to 70 cm⁻¹ for laser wavelength at about 1.4 to 2.1 micron, the absorption term exp(−dA) may be calculated at various penetration depth (d). The laser irradiance profiles (normalized by their surface value at d=0) are shown in FIG. 2, curve (1), (2) (3) for A=55 cm⁻¹ and at a focal point of f=(0.8t, 0.9t, t); and curve (4), (5), (6) for A=30 cm⁻¹, at f=(1.6t, 1.84t, 2.0t) or f=(08, 0.92, 1.0) mm, with t being the corneal total thickness (assumed to be 0.5 mm). Also shown in FIG. 2 is the profile for a non-focused case, curve (7), having its maximal near the corneal surface.

Several important features may be addressed based on the calculated profiles shown in FIG. 2. The peak irradiance (P*) of a focused laser is inverse proportional to the absorption coefficient (A), that is a laser having a larger A value curve (4) has a lower P* than smaller A value laser curve (1). However, this peak power effect from focusing factor (B) may be totally suppressed by the exponentially decreasing term by an appropriate choice of the focal length (f). Mathematically, a perfect flat-top (PFT) profile would be possible under the condition of B=exp(+dA) for all depth (d). For A=55 cm⁻¹ as an example, the PFT condition is given by B=(1.0, 3.96, 7.9, 15.7) at depth position of d=(0, 0.25, 0.375, 0.5) mm. This PFT condition is fundamentally impossible to meet under a typical lens design having B=(1, 4, 7.1, 16) for spot size of (1, 0.5, 0.37, 0.25) R, R being the spot size on the corneal surface which produces an almost flat-top (AFT) oscillating function as shown by curve (3) for A=55 cm⁻¹ and curve (5) and (6), for smaller A=30 cm⁻¹. These AFT profiles require a deeper focal point (f) at about 1.0 mm (or two times of the corneal thickness) for the case of A=30 (1/cm), but not for higher A values. In general, the condition for AFT profile is given by B*=exp (f*A), or f*=(lnB*)/A, therefore, f*=(1.4, 0.92, 0.7, 0.62, 0.55, 0.46) mm, for A=(20, 30, 40, 45, 55, 60) cm⁻¹ and B*=16. As shown by curve (3) and (5), a laser at 1450 nm (with A=30 cm⁻¹) should be focused at about two times of the corneal thickness versus about corneal thickness (0.5 mm) for a laser at about 1890 nm (with A=55 cm⁻¹).

The laser irradiance profile is almost flat in the vertical (depth) direction, the laser footprint (spot size) is a conical shape in a focused beam. Therefore, there is no homogeneous laser irradiance profile exist in both vertical and horizontal directions. The most one may achieve is an approximate flat-top (AFT) profile in the depth direction and under the condition of AFT defined earlier. To achieve maximal thermal shrinkage tissue volume (MTTV), the preferred focal length includes f=f*+/−0.2 mm for small A=(20 to 55) cm⁻¹, and f=f*+/−0.05 mm for large A=(56 to 70) cm⁻¹ with f*=(0.4-1.4) mm, as demonstrated by FIG. 2. Therefore, A=(20 to 55) cm⁻¹ or laser at (1400 to 1500) nm, (1860 to 1890) nm and (2050 to 2150) nm are the most preferable spectra which show less sensitivity in laser profile to the focal length than that of 1890 to 1920 nm having higher A=(55 to 70) cm⁻¹, as shown by curves in FIG. 2.

The currently used HLTK and conventional DTK devices still suffer instability, poor predictability and postoperative regression which may be significantly reduced by choosing an optimal focusing depth associated to the absorption coefficient (A) at a given laser spectrum, to be further detailed as follows.

The threshold temperature (T*) for permanent thermal shrinkage (PTT) of the treated tissue is about 58 degree C. It is important to note that PTT occurs near the surface layer of the treated area in a collimated beam, whereas it penetrates deeply into the area defined by the focal point (or focal length f) in a focused beam. Deep penetration is one of the critical element for efficacy and postoperative stability proposed in this invention. Prior art of Sand suffers major regression due to its non-contact, non-focused superficial laser heating. The proposed focused mode in this invention overcomes the drawback of prior art and also provides other advantages including less damage to the corneal epithelial and endothelial layers, while having sufficient coagulation in almost the entire stroma versus only less than 25% in non-focused cases. The prior art of Sand using a non-focused, short pulsed Ho:YAG laser suffers epithelial damage (since the laser power peaked on the surface) and insufficient coagulation in deep stroma. The currently used commercial DTK laser using tightly focused CW diode laser (with f about 0.3 to 0.5 mm) had improved the penetration depth, however, it still has a narrow peak profile which results in postoperative regression and low initial efficacy, less than 2.0 diopter correction. The broader laser profile under the AFT condition disclosed in this invention not only provides the efficacy (for higher diopter hyperopia correction, say up to +5 diopter) but also reduces the postoperative regression.

As shown in the book of Atchinson (p. 110), the transmittance curve (TC) in ocular tissue is very sensitive to laser spectrum, particularly in the range of 1.8 to 1.9 micron, changing from about 40% to 1% (at 1.9 micron). Therefore it is also important to specify the diode laser spectrum with a tolerance of about 2 mm (for 1.85 to 2.0 micron range), or 20 mm (for 1.4 to 1.5 micron range), where the latter spectrum having much wider tolerance is the most preferable embodiment of this invention, because a typical diode laser has a tolerance about 5 to 10 nm.

Penetration Depth and Temperature Profile

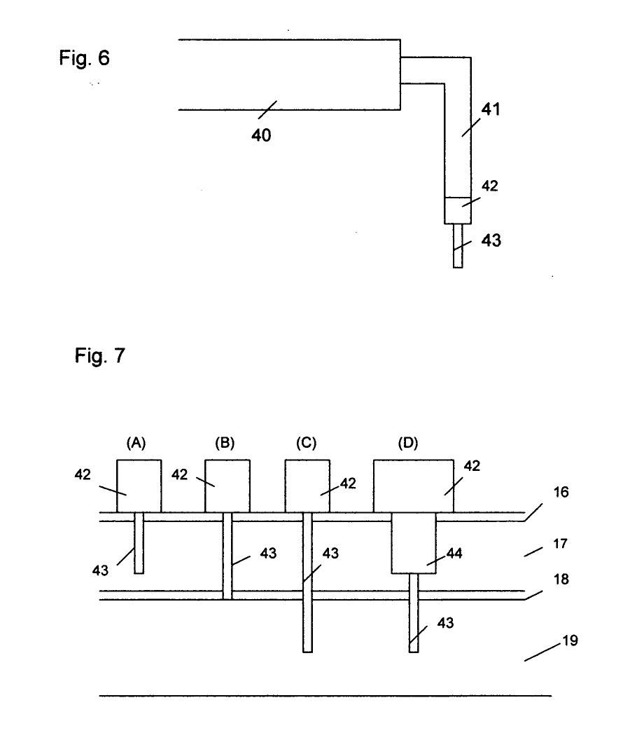

For RF devices, the penetration depth is controlled by the configuration of the electrode tip (to be shown later in FIGS. 6 and 7). For lasers, the penetration depth is governed by the power, spot size and spectrum of the laser. The proposed visible lasers (about 0.45 to 0.78 microns) have an absorption coefficiency (A) about 400 to 1000 (1/cm) in melanin and about 10 to 300 (1/cm) in hemoglobin (Hb), oxy or deoxy. For example, penetration depth about (defined by remaining power of about 3% after absorption) 0.35 mm and 1.2 mm for A equals 100 1/cm and 50 1/cm, respectively. These penetration depth range of 0.3 to 0.6 mm in choroids or ciliary body (in zone-2) are the criteria for our selection of visible laser spectra proposed in the present invention.

On the other hand, for IR laser of 1.4 to 2.2 microns, the absorption (0.5 mm path) in cornea, sclera or conjunctival tissue is about (from Atchison and Smith, Optics of the Human Eye, Butterworth-Heinemann, 2000, p. 110) 60% (at 1.4 micron), 75% (at 1.45, 1.87 and 2.1 micron), 50% (at 1.8 micron), 95% (at about 1.89 micron) and over 99% (at about 1.92 microns) for non-focused beam. These are the criteria for the selected IR lasers in addition to the focusing factor (B) discussed earlier. The absorption depth at various laser spectra for various ocular tissues is one of the critical elements in defining system parameters of this invention. Given A value of the treated tissue, we may predict the penetration depth (d) by the revised Beer's law, Bexp(-dA) as discussed earlier. However, the penetration depth is also an increasing function of the power of the thermal energy beam, laser or RF wave.

As shown earlier (FIG. 2), higher A value tends to move the temperature peak profile forward the corneal epithelium. Therefore, to avoid cornea epithelial damage while achieving significant deep thermal penetration, the laser wavelength and the focal length must be carefully specified. The most preferred laser wavelength disclosed in this invention is much narrower than that of prior art of Sand, 2.0 to 2.2 micron.

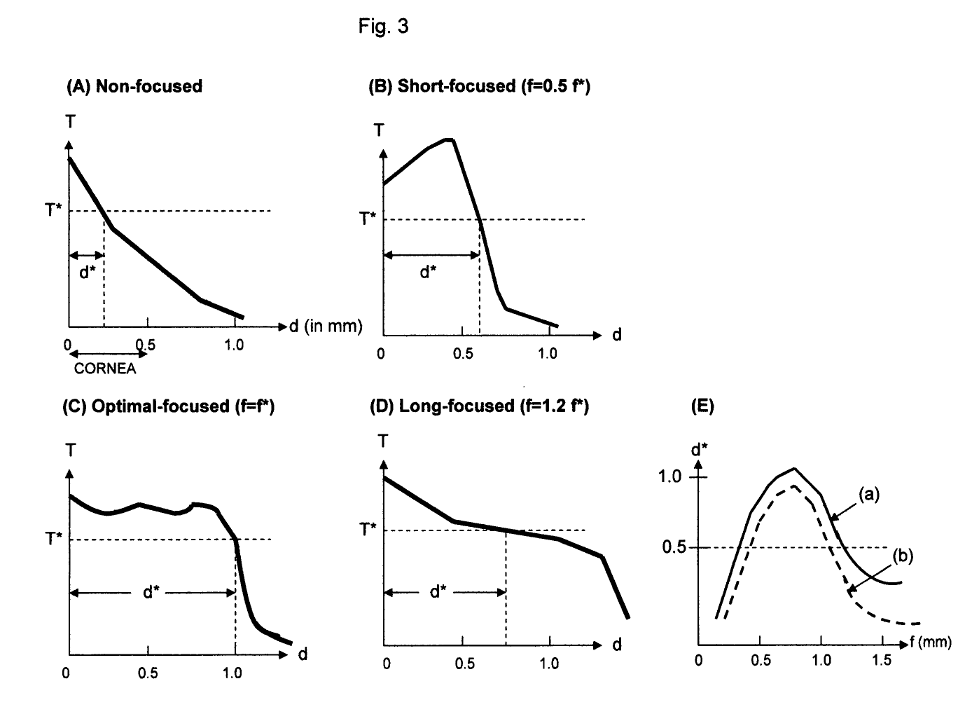

As shown in FIG. 3, the temperature profile of the treated tissue is schematically shown (relative to the surface temperature) based on the calculated laser power density profiles shown earlier in FIG. 2. The profiles shown by FIG. 3(A) to (D) are for non-focused, short-focused (f<f*), optimally focused (with f=f*) and long focused (f>f*) cases, respectively, where f* is defined by B*=exp(f*A), or f*=(lnB*/A). where B*=(7-16) is the value at focal point, depending on the spot size at the focal point, (0.08-0.5) mm.

It was shown (Stringer HPT, Nature, vol. 204, p. 1307, 1964) that stroma collagen shrinkage temperature starts at about 58° C. (defined as the threshold temperature T*) to 75° C. Given the T*, we may evaluate the effective penetration depth (d*), the depth where tissue thermal shrinkage starts to effectively occur. As shown by FIG. 3(A) to (D), d* increases with f up to about f=f*, then it starts to decrease and approach the non-focused value. FIG. 3(E) shows d* versus the focal length (f) for two cases: (a) for T*=0.6Ts and (b) T*=0.8Ts, where Ts is the surface temperature. The preferred d* is about 0.3 to 1.0 mm and most preferable about 0.4 to 0.6 mm.

The significance of FIG. 3 may be summarized as follows: (a) non-focused laser has the shortest effective penetration, d*=(0.1-0.2) mm depending on A and T*; (b) short focused case with f<f* has lower temperature on the treated surface, or less risk of surface damage, which, however, has higher temperature nearly the endothelial layer (about 0.5 mm for the case of zone-1 treatment) and higher risk of endothelial damage; (c) the optimal focusing case (f=f*), the temperature profile is almost flat-top, therefore it has the maximal d*, whereas the risk of epithelial or endothelial layers may be reduced under controlled laser power and treatment (exposure) time, typically about 1 to 5 seconds; (d) for long focused beam (with f>f*), the value of d* decreases. To keep d* at least 80% of the corneal stroma thickness (0.5 mm), as an example, the preferred parameters of this invention includes the focal length (f) about (0.5-3.0) mm and most preferable (0.6-2.0) mm, or about (0.9-1.2) times of f, where f* is about 0.4 to 1.4 mm, for B*=(10-16) The preferred range of focal length also significantly reduces the risk of overheating of the epi- or endo-thelial layer of the cornea.

It is important to emphasize that typical value of f*=(0.4-1.4) mm for absorption coefficient A=(20-70) cm⁻¹, therefore the tolerance of f, given by (0.8-1.4) f*, is only about (0.05-1.5) mm. Considering the lens design manufacturing accuracy for the focal length and surgeon's control of the tip of contact hand piece, limited to not better than 0.05 mm, the most preferred embodiment of this invention includes the use of A=(20-55) cm⁻¹, or laser spectrum about (all in mm) (1400-1550), (1875-1885), (2050-2150) which allows a reasonable tolerance control of f to be about (0.1-0.3) mm, and f*=(0.41-1.4) mm for B*=16, as an example.

The next preferred laser spectral range is (1885-1900) and (2000-2040) nm having A=(56-70) cm⁻¹, which however has a smaller tolerance only about (0.05-0.1)mm. Laser spectrum having A value much larger than 70 cm⁻¹, such as (2700-3200) nm proposed in the prior arts of Lin-62-68 should be avoided according to the teaching of this invention.

Above analysis covers the treatments in using lasers in the IR ranges, (1.4-2.2) microns, where the treated ocular tissues are cornea or sclera. For thermal shrinkage of choroids or ciliary-body using visible laser of (0.5-0.78) microns, the absorption coefficient A is about (20-85) cm⁻¹ (after: Geeraels and Berry, Am J. Ophthal. Vol. 66, pp. 15-20, 1968, see also FIG. 14.2a in a book by Atchinson and Smith: Optics of the human eye (Butherworth-Heinemann, chapt. 14, 2000). Therefore, our analysis based on the theory of revised Beer's law applies to both IR and visible lasers with the spectra specified in the preferred embodiment of this invention.

The unique features and teaching disclosed by this invention, including the preferred embodiments for laser parameters (power, spot size and spectrum) and lens design (focal length and spot size control configurations), offer both technical and clinical advantages over prior arts. The methods and apparatus disclosed based on the new theoretical formulas and lens design (to be detailed next) in this invention are not available by prior arts.

Lens Design

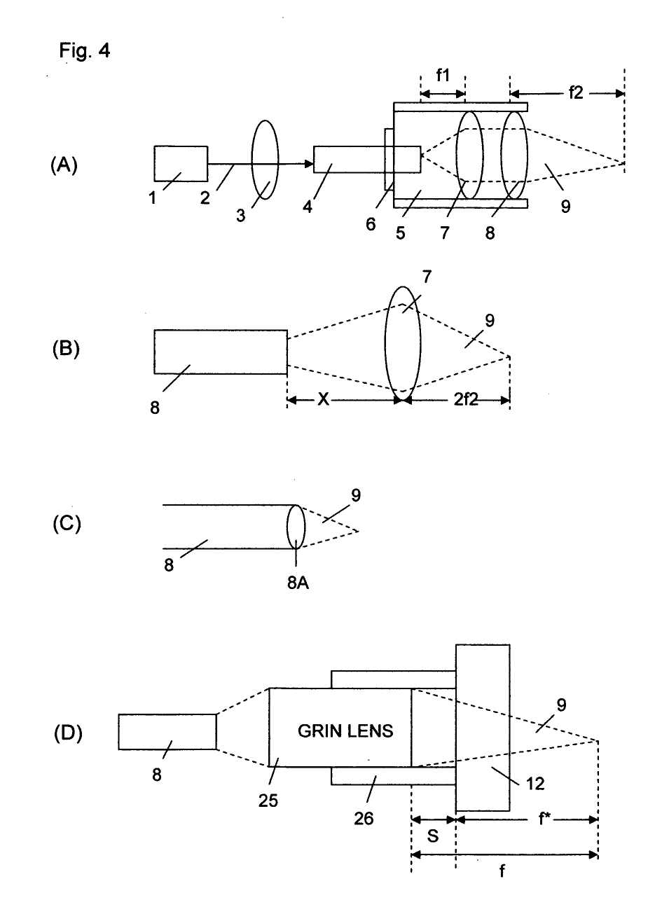

Based on the above discussed laser and temperature profiles of focused laser, the control means of laser spot size and penetration depth includes the following preferred configurations. As shown in FIG. 4(A), the basic laser 1 having IR or visible output 2 specified earlier is coupled by a lens 3 to an optical fiber 4 which is further connected by a connector 6 (the commercial SMA adaptor) to a hand piece 5 having a pair of lens 7 and 8 to produce a focused beam 9 penetrating into the treated ocular tissue (cornea, sclera, choroids or ciliary body). By positioning lens 7 at a distance of its focal point (f1) away from the end face of fiber 4, a collimated beam is produced and then focused by the second lens 8 having a focal length of f2 which is defined by the revised Beer's law discussed earlier. For example, the preferred embodiment of this invention includes a fiber core diameter about (0.1-1.0) mm having a numerical aperture (NA) about (0.15-0.35) and focal length f1 about (0.5-5.0) mm. For best clinical outcome in zone-1 treatment for cornea thickness about 0.5 mm, the Beer's law calculations (shown in FIG. 2) give us the preferred focal length, f2 about (0.5-4.0) mm for absorption coefficient A=(20-70) cm⁻¹, and most preferable about (0.8-2.5) mm for A=(20-55) cm⁻¹, as discussed earlier. The laser spot size on the treated ocular surface controlled by the first lens 7 is about R1=(0.8-2.0) mm which is focused to a spot size about R2=(0.08-0.5)mm, most preferable about (0.1-0.3)mm, at the focal point. The above preferred parameters of f1, f2, R1 and R2 provide us the maximal tissue thermal shrinkage volume and penetration depth to achieve efficient shrinkage of the treated areas, whereas the risk of epithelial or endothelial layer of the cornea, as an example, is minimized. The preferred lens of 7 and 8 include spherical or aspherical lens having a configuration of plano-convex or biconvex.

FIG. 4(B) shows another preferred embodiment of this invention, where the lens 7 having an effective focal length of f1 (for front surface) and f2 (for back surface) such that the minimal spot size at a position 2(f2) can be controlled to the range of (0.08-0.5) mm by choosing f1 and f2 and adjusting the distance X. For example, minimal spot size at 2(f2) of 0.1 mm can be achieved by X=2(f2) for a fiber core diameter of 0.1 mm. The preferred value of f2 is similar to the lens 8 of FIG. 4(A). Alternatively, as shown in FIG. 4(C), the fiber end 8-A may be a curved surface to produce a focused beam 9 without the use of lens 7 or 8.

Another preferred embodiment shown by FIG. 4(D) is to use a non-contact graded index (GRIN) lens 25 (commercially available) to couple the output from the fiber 8 and refocused by the output end of the GRIN lens having a focal length f=S+f* where S is an adjustable distance between the GRIN lens output end and the surface of the treated area (12) controlled by a holder (26). For example, for thermal shrinkage of cornea stroma (with a thickness of 0.5 mm), the preferred parameters are S=1.3 mm, for f*=0.98 mm and at a given GRIN lens focal length (in air) of about f=2.0 mm, note that a 1.4 factor is needed to convert the focal length in air to that inside the cornea having a refraction index about 1.4, that is f*=(2.0−1.3)×1.4=0.98 mm.

The contact mode shown by FIG. 4(A) to (C) may be revised easily to a non-contact mode (or configuration) by attaching an extra lens holder (26) as shown in FIG. 4(D). We also note that the focal length in cornea is about 1.4 times of the focal length in air for a given lens due to the higher refraction index of 1.37 in cornea. This is another important lens design factor which cannot be ignored.

One-Zone and Two-Zone Thermal Pattern

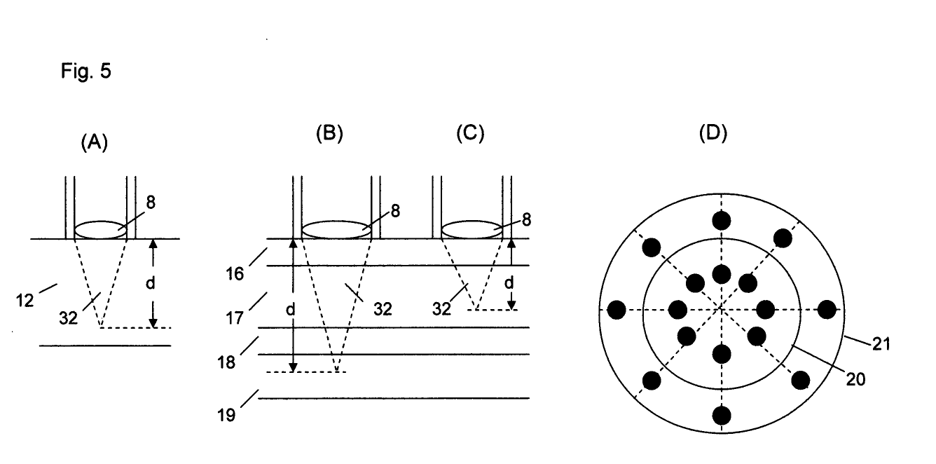

FIG. 5 (A) to (C) illustrate examples of the preferred embodiments in this invention. As shown in FIG. 5(A), the corneal tissue 12 is heated by a focused laser beam from a lens 8 which can be contacted or non-contacted to the corneal surface. The preferred laser heated area 32 includes a depth (d) about 80% to 90% of cornea thickness (about 0.5 mm). The preferred laser includes laser having a wavelength about 1.4 to 2.2 micron, most preferable about 1.45, 1.88 and 2.1 microns with cornea absorption coefficient (A) about 20 to 70 cm⁻¹, most preferable of 25 to 55 cm⁻¹ (after: Atchinson and Smith, Optics of human eye, p. 108), which gives an absorption of about 70% to 95% at a depth of 0.5 mm. This is based on Beer's law T=1-exp (−Ad), and the published data of Transmittance (T) in the text of Atchinson. Higher A value tends to move the temperature peak profile forward the corneal epitheliums. Therefore, the laser wavelength must be carefully specified to avoid epithelial damage but deep enough (about 450 micron) thermal penetration. The most preferred laser wavelength disclosed in this invention is more specific than that of prior art of Sand, 2.0 to 2.2 micron.

FIG. 5-B shows another preferred embodiment with the energy beam focused by the lens 8 into ciliary body (CB) layer 19 with a heated area 32, where the preferred laser spectra include visible lasers which are mainly absorbed by pigments (or melanin) and blood cells of choroids and CB. Green laser from second harmonic of Nd:YAG, Nd:YLF or visible lasers about 0.48 to 0.78 microns are preferred, because of their high transparency of conjunctiva and scieral layer (16, 17) and strong absorption in the CB 19 and choroids layer 18. The heated area 32 includes a penetration depth (d) about 0.5 to 1.2 mm.

FIG. 5-C shows a focused laser having a wavelength in IR of about 1.4 to 2.1 microns with a strong absorption by the conjunctiva 16 or sclera tissue 17. Another preferred embodiment for the laser beam is to use a fiber with the fiber tip contacting the treated surface. For the fiber-delivered contact method, fiber materials used shall be highly transparent at the selected laser spectra. We note that IR lasers at 1.4 to 2.2 microns used in 5-A and 5-C shall be excluded in 5-B due to their strong absorption in sclera and conjunctiva. Fiber tip size of about (0.5-1.0) mm having a round end surface is preferred for deep penetration, where the laser beam is focused into the treated area.

The thermal pattern is shown in FIG. 5-D, where the preferred heating area includes zone-1 circle 20 having a diameter about 6 to 8 mm, and zone-2 circle 21 about 10 to 14 mm in diameter (outside the limbus). The preferred pattern also includes at least 4 spots, most preferable about 8 to 32 spots, in each of the treated zones and the number of spots proportional to the desired diopter of hyperopia or presbyopia correction.

The preferred temperature of the treated area is about (50-80) degree Celsius, most preferable about (58-75) degree Celsius, but below the tissue damage temperature. The temperature increases of the treated tissue may be controlled by the average power (P) and spot size of the energy beam. The preferred examples includes, for spot size of (0.5-1.0) mm, P=(5-200) mW for visible lasers and P=(100-500) mW in infrared lasers. These preferred power will be doubled if the spot size increases by a factor of 1.4. Therefore, for spot size (at the treated area) range of (0.2-1.5) mm, the preferred range is about P=(0.05-2.0)W depending also on the laser spectra and types of tissue heated (conjunctiva, sclera, choroids or ciliary body).

We note that the required power for efficient thermal and shrinkage of treated tissue by visible laser is lower than that of infrared due to the strong absorption of visible laser in choroids and ciliary body. The earlier discussion clearly demonstrate that depending on the types of tissues to be heated, the laser parameters must be specified accordingly. Without choosing the appropriate laser parameters, localized heating which causes the thermal shrinkage of the selected areas will fail.

Presbyopia Correction

In the prior arts of HLTK, DTK or CK for the treatment of hyperopia, the treated area is in the cornea are defined as one-zone method in comparing to the two-zone method which also includes the second zone in the sciera area (outside the limbus) as proposed in the present invention. As shown in FIG. 1, prior arts cause the change of the cornea central surface only by the shrinkage of zone-1, an area defined by a diameter about 6 to 8 mm (within the limbus). Therefore, their treatments are limited to low hyperopia correction, up to about 2 diopters (after regression). By additional thermal shrinkage in zone-2, outside the limbus about 10 to 14 mm in diameter, one shall expect a 50% to 100% extra efficacy in hyperopia correction, in addition to the reduction of postoperative regression.

The shrinkage of the treated zone-2 area in conjunctiva, sclera, choroids or CB (as shown in FIG. 5) will result in two effects detailed as follows. First, the displacement of lumbal tissue and/or sclera tissue away from the lens will result in further bulging of the corneal central surface and therefore it adds extra effect on hyperopia correction to the treated zone-1 area (on the cornea). Second, the shrinkage of treated areas in zone-2 will result in either lens anterior shift or the loosening of the ciliary-body-zonule complex, therefore accommodative ability of the lens increases in presbyopia. The two-zone method disclosed in the present invention provides effective treatment of presbyopia, in addition to the enhancement of hyperopia correction.

Two-Zone Method using RF Wave

In addition to the thermal energy from the lasers described above, the present invention also discloses the use of RF wave device. As shown by FIG. 6 for the zone-two treatment defined earlier. A typical RF device consists of a RF generator (not shown) and a hand piece 40 which is connected to an end piece 41, an insulator 42 and a conductor tip 43. The preferred embodiments of the penetration of the tip 43 shown in FIG. 7(A) to (C) for various penetration depths in the sclera 17, choroids 18 and ciliary body 19, are controlled by the length of the conductor tip 43. The larger diameter of the insulator 42 is used as a “stopper” for the penetration depth of tip 43. FIG. 7(D) shows another preferred embodiment having two-sector of insulator 42 and 44, such that only the choroids and ciliary-body 19 is heated, while the sclera 17 and conjunctiva 16 are kept un-heated. Similar device of FIG. 7(A) may be used for the treatment of zone-1 to control the penetration depth of the cornea tissue for hyperopia correction, having a preferred depth about 450 micron, versus about 0.5 to 1.2 mm depth for presbyopia treatment in zone-2.

The preferred thermal patterns generated from a laser or a RF device shall include ring spots and any unspecified symmetric patterns within the region defined by a radial distance of from about 6 mm to about 8 mm from the center of the cornea (for zone-1) and about 10 to 14 mm (for zone-2).

There are no commercially available systems available so far using a two-zone method or a one-zone method (under the optimal focusing condition) as disclosed in this invention, due to the lack of empirical data and teaching disclosed in this invention.

It has been clinically shown that sclera expansion (by SEB) of Schachar or laser sclera ablation (Lin's prior art) may reduce the intraocular pressure (IOP) particular for subject with elevated IOP. Therefore the thermal method disclosed in this invention shall achieve the same. The IOP reduction may be resulted from the increase of the pore size in the trabecular meshwork after tissue the shrinkage particular in the zone-two treatment.

The preferred embodiments for laser energy delivery of this invention also include: a computer controlled scanning means such as motorized galvometer, and delivery means of articulated arm or optical fiber. The scanning means may be further integrated to a slip lamp and the treating thermal pattern, penetration depth and laser spot size may be controlled by software, where the laser energy is delivered to the treated area by a non-contact mode versus the contact mode when an optical fiber and hand piece are used. In addition, the ablation patterns proposed in this invention may be produced by software, motorized device or manually.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

I claim:

1. A method of thermal shrinkage of ocular tissue comprising the steps of:
(a) selecting a thermal energy beam having a predetermined power, spot size, penetration depth and wavelength; and
(b) delivering said thermal energy beam to said ocular tissue in a predetermined pattern and area of an eye, whereby patient's hyperopia is corrected, or accommodation for near vision is improved.

2. A method of claim 1, wherein said ocular tissue includes cornea, sclera, choroids or ciliary-body of an eye within a circle area having a diameter of about 6 to 8 mm defined as zone-1, or about 10 to 14 mm defined as zone-2.

3. A method of claim 1, wherein said accommodation is improved by the change of the elastic property or the available spacing of the sclera-ciliary-zonule complex resulted from said thermal shrinkage of said ocular tissue in zone-2 defined in claim 2.

4. A method of claim 1, wherein said accommodation is caused by the combined effect of axial movement and surface curvatures change of the crystalline lens of an eye.

5. A method of claim 1, wherein hyperopia is corrected via the shrinkage of corneal stroma in zone-1 and enhanced by the shrinkage of said ocular tissue in zone-2.

6. A method of claim 1, wherein said energy beam includes a laser having a wavelength of about (0.48-2.2) micron, a spot size about R1=(0.8-2.0) mm on the treated ocular surface, and a focused minimal spot size about R2=(0.08-0.5) mm inside said ocular tissue.

7. A method of claim 1, wherein said predetermined penetration depth (d) of said energy beam is governed by a normalized laser power density equation P=Bexp(−dA), where the absorption coefficient of said ocular tissue at said predetermined laser wavelength and includes a preferred value of A=(20-70) cm⁻¹, most preferable (20-55) cm⁻¹; B is a focusing factor having a maximum value at the focal point about B*=(7-16) given by the square of (R1/R2) with R1 and R2 defined in claim 6.

8. A method of claim 1, wherein said energy beam is delivered to the predetermined area zone-1 or zone-2 defined in claim 2 by an optical fiber which is further connected to a hand piece and coupled to at least one focusing optics including spherical, aspherical, cylindrical or graded-index (GRIN) lens.

9. A method of claim 8, wherein said focusing optics includes a focal length (f1) about 0.8 to 1.4 times of f*, when it is contacted to said ocular tissue surface; or a focal length of f1+S, when it is used in a non-contact mode having a distance S away from the ocular surface; where f*=(lnB*)/A is an optimal focal length about 0.4 to 1.4 mm for the preferred A=(20-70) cm⁻¹ and B*=16.

10. A method of claim 6, wherein said laser includes visible laser of argon ion laser at (488-514) nm, frequency-doubled YAG laser at 526 and 532 nm, He—Ne laser at 633 nm, krypton-ion laser at 647 nm, dye laser at (0.6-0.7) micron, or diode lasers at about (0.63-0.78) micron, where said visible laser is used to cause thermal shrinkage of choroids or ciliary body in the predetermined area of zone-2 defined in claim 2 for the treatment of presbyopia.

11. A method of claim 1, wherein said laser includes infrared laser having an ocular tissue absorption coefficient (A) about (20-70) cm⁻¹ or a wavelength of about (1.4-2.2) microns, most preferable of A=(20-55) cm⁻¹ or a wavelength of about (1400-1500) nm, (1860-1890) nm or (2050-2150) nm, where said infrared laser is used to cause thermal shrinkage of the corneal stroma in zone-1 or sclera in zone-2, the predetermined area defined in claim 2 for the treatment of hyperopia or mono-vision presbyopia.

12. A method of claim 11, wherein said laser includes semiconductor diode laser at (1.4-1.9) microns, Ho:YAG laser at about 2.1 microns, Nd:YAG laser at about 1.4 micron, diode-pumped fiber laser at about (1.4-1.5) micron or Nd:glass laser at about 1.54 micron, operated at free running long pulse (longer than 500 microseconds) or continuous wave (CW) and power of about (0.05-2.0) W at said predetermined area of an eye.

13. A method of claim 1, wherein said energy beam includes a radio frequency wave at about (200-500) KHz and power of about (0.5-5.0) W.

14. A method of claim 1, wherein said energy beam includes radio frequency wave generated from an electrode device, a bipolar device, or a plasma assisted electrode device, having a hand-piece connected to an insulator and a conductor tip, where the conductor tip includes a length of about (0.45-1.2) mm penetrated to corneal stroma in zone-1 area for hyperopia correction, or to sclera choroids or ciliary body in zone-2 area for hyperopia enhancement or presbyopia correction.

15. A method of claim 1, wherein said predetermined pattern includes radial ring spots or any non-specific shapes, generated manually or by a computer software, where the preferred number of spot includes about (8-32) spots in each of the predetermined zone-1 or zone-2 area.

16. A method of claim 1, wherein said energy beam is delivered to said predetermined area to cause a localized temperature preferred to be about (55-85) degree Celsius, most preferable about (58-75) degree Celsius, and an effective penetration depth of about (0.3-1.0) mm defined by a depth range in which the ocular tissue temperature is above the shrinkage threshold, about 58 degree Celsius.

17. A system for the treatment of presbyopia or hyperopia consisting of
(a) a thermal energy beam having a predetermined power, spot size, penetration depth and wavelength; and
(b) a delivering means to deliver said energy beam to the ocular tissue in a predetermined pattern and area of an eye.

18. A system of claim 17, wherein said ocular tissue includes cornea, sclera, choroids or ciliary-body of an eye within the region defined by a circle having a diameter of about 6 to 8 mm (zone-1) or about 10 to 14 mm (zone-2).

19. A system of claim 17, wherein said presbyopia is treated by the increase of accommodation due to lens axial movement or lens curvatures change caused by the thermal shrinkage of said ocular tissue in zone-2 defined in claim 18; and said hyperopia is corrected via the corneal stroma shrinkage in zone-1 and enhanced by said ocular tissue shrinkage in zone-2.

20. A system of claim 1, wherein said energy beam includes a laser having a wavelength of about (0.48-2.2) micron, a spot size about R1=(0.8-2.0) mm on the treated ocular surface, and a focused minimal spot size about R2=(0.08-0.5) mm inside said ocular tissue.

21. A system of claim 17, wherein said energy beam is delivered to the predetermined area zone-1 or zone-2 defined in claim 19 by an optical fiber which is further connected to a hand piece and coupled to at least one focusing optics including spherical, aspherical, cylindrical or graded-index (GRIN) lens.

22. A system of claim 21, wherein said focusing optics includes a preferred focal length (f1) about 0.8 to 1.4 times of f*, when it is contacted to the surface of said ocular tissue; or about f1+S, when it is used in a non-contact mode having a distance S away from the ocular surface; where f*=(lnB*)/A is an optimal focal length about 0.4 to 1.4 mm for the preferred absorption coefficient A=(20-70) cm⁻¹ and B*=16.

23. A system of claim 20, wherein said laser includes visible laser of argon ion laser at about (488-514) nm, frequency-doubled YAG laser at 532 and 526 nm, He—Ne laser at 633 nm, krypton-ion laser at 647 nm, dye laser at (0.6-0.7) micron, or diode lasers at about (0.63-0.78) micron, where the visible laser is used to cause thermal shrinkage of choroids or ciliary body in the predetermined area of zone-2 defined in claim 18 for the treatment of presbyopia.

24. A system of claim 20, wherein said laser includes infrared laser having an ocular tissue absorption coefficient (A) about (20-70) cm⁻¹ or a wavelength of about (1.4-2.2) microns, most preferable of A=(20-55) cm⁻¹ or a wavelength of about (1400-1500) nm, (1860-1890) nm or (2050-2150) nm, where said infrared laser is used to cause thermal shrinkage of the corneal stroma in zone-1 or sclera in zone-2, the predetermined area defined in claim 18 for the treatment of hyperopia or mono-vision presbyopia.

25. A system of claim 20, wherein said laser includes semiconductor diode laser at (1.4-1.9) microns, Ho:YAG laser at about 2.1 microns, Nd:YAG laser at about 1.4 micron, diode-pumped fiber laser at about (1.4-1.5) micron, or Nd:glass laser at about 1.54 micron, operated at free running long pulse (longer than 500 microseconds) or continuous wave (CW) and power of about (0.05-2.0) W at said predetermined area of an eye.

26. A system of claim 17, wherein said energy beam includes a radio frequency wave at about (200-500) KHz and power of about (0.5-5.0) W.

27. A system of claim 17 wherein said energy beam includes radio frequency wave generated from an electrode device, a bipolar device, or a plasma assisted electrode device, having a hand-piece connected to an insulator and a conductor tip, where the conductor tip includes a length of about (0.45-1.2) mm penetrated to corneal stroma in zone-1 area for hyperopia correction, or to sclera choroids or ciliary body in zone-2 area for hyperopia enhancement or presbyopia correction.
